(19) United States
(12) Patent Application Publication
(10) Pub. No.: US 2024/0043357 A1
(43) Pub. Date: Feb. 8, 2024

(54) METHOD FOR THE SELECTIVE HYDROGENATION OF THE C2 FRACTION COMPRISING ACETYLENE IN THE PRESENCE OF A CATALYST IN MONOLITHIC FORM

(71) Applicant: IFP Energies nouvelles, Rueil-Malmaison Cedex (FR)

(72) Inventors: Florent ALLAIN, Rueil-Malmaison Cedex (FR); Yacine HAROUN, Rueil-Malmaison Cedex (FR); Marion SERVEL, Rueil-Malmaison Cedex (FR)

(73) Assignee: IFP Energies nouvelles, Rueil-Malmaison (FR)

(21) Appl. No.: 18/266,062

(22) PCT Filed: Nov. 30, 2021
(86) PCT No.: PCT/EP2021/083464

§ 371 Date: Jun. 8, 2023

(30) Foreign Application Priority Data
Dec. 10, 2020 (FR) FR2013013

(51) Int. Cl. C07C 5/09; B01J 35/04; B01J 35/00; B01J 23/44; B01J 35/10

(52) U.S. Cl. CPC C07C 5/09; B01J 35/04; B01J 35/0066; B01J 23/44; B01J 35/1004; C07C 2523/44; C07C 2523/42; C07C 2523/755

(57) ABSTRACT

A method for selective hydrogenation of a C2 steam cracking fraction comprising acetylene, in the presence of a catalyst comprising an active phase based on at least one group VIII metal and a support provided in the form of a ceramic or metal monolith, characterized in that said support comprises a number of channels per unit length, CPSI, of between 300 and 1200, and in that the active phase is provided in the form of a layer on the walls of said support, the thickness of said layer of active phase being between 30 μm and 150 μm.

### TECHNICAL FIELD

The subject matter of the invention is a method for selective hydrogenation of polyunsaturated compounds in a hydrocarbon feedstock, more particularly in the C2 steam cracking fraction, in the presence of a catalyst provided in the form of a ceramic or metal monolith.

### STATE OF THE ART

There are a number of types of monolithic support, developed and manufactured using various techniques. The monolithic supports can be made of ceramic materials such as, for example, alumina or silicon carbide or zirconium or cordierite. There are also monolithic supports with metal materials, made for example of steel, stainless steel, and many other types of metals.

There are various ways of manufacturing the monolithic supports. The manufacturing technique is well known to the person skilled in the art and may be found in the article by Forzatti et al., “Preparation and characterization of extruded monolithic ceramic catalysts”, Catalysis Today 1998, 41, 87-94, or else in the article by Avila et al., “Monolithic reactors for environmental applications: A review on preparation technologies”, Chem. Eng. J. 2005, 109, 11-36, or, lastly, in the article by Sandeeran et al., “Preparation Methods and Their Relevance to Oxidation”, Catalysts 2017, 7, 62.

Ceramic and metal monoliths can take on different geometric shapes and sizes. They consist of parallel channels separated from one another by thin walls. These channels can have various cross-sectional shapes: rectangular, cylindrical, triangular, hexagonal, and many other, more complex shapes.

(Ceramic or metal) monolith supports are generally characterized by the density and the size of the channels, more specifically by the number of channels per unit length, which is referred to as CPSI (for “channels per square inch”). As its abbreviation indicates, it corresponds to the number of channels intercepted by a cross section of 1×1 inch, i.e., 2.54×2.54 cm. The monoliths may also be characterized by their wall thickness, or by the channel window width when the channels are rectangular or square in cross section, or else by their porosity. The porosity can be calculated by the following formula:

$$ε = 1 - \frac{{ρ}_{m}}{{ρ}_{mat}}$$

with:
ε: porosity or void ratio of the monolith;
ρₘ: density of the monolith;
ρₘₐₜ: density of the material of the monolith.

Metal or ceramic monoliths can be used in a variety of catalytic applications, particularly in the treatment of exhaust gases (US1969/3441381, US1971/35971653) or as an NOₓ reduction catalyst (Tomasic, V. 2007), or else in selective hydrogenation of hydrocarbon feedstocks comprising polyunsaturated compounds.

The review article “Selective hydrogenation of 1,3-butadiene in the presence of 1-butene under liquid phase conditions using structured catalysts”, by F. J. Mendeza et al., published in Catalysis Today, 289 (2017), 151-161, focuses on the use of a catalyst support in the form of metal monoliths or foams for the selective hydrogenation of 1,3-butadiene. This document discloses that the use of a support made of foam or of monolith coated with NiPd/(CeO₂—Al₂O₃) active phase gives good results in terms of conversion and in terms of selectivity in selective hydrogenation of 1,3-butadiene. Catalysts having a support in the form of a monolith have an active phase layer thickness of 18 μm or 20 μm.

The article “Catalyst deactivation in liquid- and gas-phase hydrogenation acetylene using a monolithic catalyst reactor” by Asplud et al., published in Catalysis Today, vol. 24 (181-187) 1995, focuses on the use of a catalyst support in the form of an α-alumina ceramic monolith for the selective hydrogenation of acetylene. This document discloses the use of a monolithic support impregnated directly with PdCl₂ on the walls of the monolith, where the active phase, obtained on the basis of palladium, has a thickness of 200 μm.

In this context, one of the objectives of the present invention is to provide a method for selective hydrogenation of a C2 steam cracking fraction, in the presence of a catalyst provided in the form of a metal or ceramic monolith supporting the active phase, which makes it possible to obtain hydrogenation performance qualities in terms of selectivity which are at least as good as, or even better than, those of the methods known from the state of the art.

The Applicant has found that a catalyst comprising an active phase based on at least one group VIII metal and a support provided in the form of a ceramic or metal monolith having a particular geometric structure, said active phase being provided in the form of a layer of defined thickness on the walls of said support, makes it possible to obtain catalytic performance qualities in terms of selectivity which are at least as good, or even improved, when it is employed in a method for selective hydrogenation of a C2 steam cracking fraction containing acetylene, and does so while reducing—even at the same conversion—the catalyst volume available for the feedstock, while limiting pressure drops.

### SUBJECT MATTER OF THE INVENTION

The subject of the present invention is a method for selective hydrogenation of a C2 steam cracking fraction comprising acetylene, said method being carried out in gaseous phase at a temperature of between 0° C. and 300° C., at a pressure of between 0.1 MPa and 6.0 MPa, at a molar ratio of hydrogen/(polyunsaturated compounds for hydrogenation) of between 0.5 and 1000, and at an hourly space velocity (HSV) of between 100 h⁻¹ and 60 000 h⁻¹, preferably between 500 h⁻¹ and 30 000 h⁻¹, n the presence of a catalyst comprising, preferably consisting of, an active phase based on at least one group VIII metal and a support provided in the form of a ceramic or metal monolith, characterized in that said support comprises a number of channels per unit length (CPSI) of between 300 and 1200, and in that the active phase is provided in the form of a layer on the walls of said support, the thickness of said layer of active phase being between 30 μm and 150 μm.

The reason is that the Applicant has observed that the deployment of such a catalyst, featuring a support in the form of a ceramic or metal monolith, said support comprising a specific number of channels per unit length, coupled with a specific thickness of the layer of the active phase on the walls of the support, makes it possible, for the same conversion, to reduce the volume of catalyst bed required for performing a selective hydrogenation reaction on a C2 steam cracking fraction comprising acetylene, while significantly improving the selectivity of the reaction for the desired products.

Preferably, said catalyst comprises a geometric surface area of between 1500 m²/m³ and 5000 m²/m³.

Preferably, the thickness of the wall of the catalyst is between 0.08 mm and 0.5 mm.

Preferably, the degree of porosity of said catalyst is between 20% and 90%.

Preferably, the thickness of said layer of active phase is between 60 μm and 100 μm.

In one embodiment according to the invention, the support is a metal monolith chosen from monoliths made of steel, stainless steel (316L, 310SS), nickel, aluminum, iron, copper, nickel-chromium, nickel-chromium-aluminum, iron-chromium-aluminum, and Inconel®.

In one embodiment according to the invention, the support is a ceramic monolith chosen from monoliths made of alumina (Al₂O₃), silica-alumina, silicon carbide (SiC), phosphorus-alumina, magnesia (MgO), zinc oxide, zirconium oxide (ZrO₂), and cordierite (Al₃Mg₂AlSi₅O₁₈).

Preferably, said group VIII metal is chosen from nickel, platinum, and palladium. More preferentially, said group VIII metal is palladium.

In one embodiment according to the invention, when the group VIII metal is palladium, the palladium content is between 0.005% and 2% by weight of said element with respect to the total weight of the catalyst.

Preferably, the number of channels per unit length (CPSI) of said support is between 400 and 700.

### DETAILED DESCRIPTION

### Definitions

In the text hereinbelow, the groups of chemical elements are given according to the CAS classification (CRC Handbook of Chemistry and Physics, published by CRC Press, editor-in-chief D. R. Lide, 81^{st} edition, 2000-2001). For example, group VIII according to the CAS classification corresponds to the metals of columns 8, 9 and 10 according to the new IUPAC classification.

The textural and structural properties of the support and of the catalyst described below are determined by the characterization methods known to a person skilled in the art. The total pore volume and the pore distribution are determined in the present invention by nitrogen porosimetry as described in the work “Adsorption by Powders and Porous Solids. Principles, Methodology and Applications”, written by F. Rouquérol, J. Rouquérol and K. Sing, Academic Press, 1999.

The term “specific surface” is understood to mean the BET specific surface (S_{BET} in m²/g) determined by nitrogen adsorption in accordance with the standard ASTM D 3663-78 established from the Brunauer-Emmett-Teller method described in the journal “The Journal of the American Chemical Society”, 1938, 60, 309.

In the present specification, the monolithic supports (ceramic or metal) are characterized by the number of channels per unit length (CPSI). It should be noted that the value of the CPSI of a catalyst comprising such a monolithic support does not change whatever the thickness of the layer of the active phase of the catalyst.

The content of group VIII metal is measured by X-ray fluorescence.

### Description of the Process

Monounsaturated organic compounds such as, for example, ethylene are at the root of the manufacture of polymers, of plastics and of other chemicals having added value. These compounds are obtained from natural gas, from naphtha or from gas oil which have been treated by steam cracking or catalytic cracking processes. These processes are operated at high temperature and produce not only the desired monounsaturated compounds but also polyunsaturated organic compounds such as acetylene, or diolefinic compounds. These polyunsaturated compounds are highly reactive and result in side reactions in the polymerization units. It is thus necessary to remove them before making economic use of these fractions. Selective hydrogenation is the main treatment developed to specifically remove undesirable polyunsaturated compounds from these hydrocarbon feedstocks. It enables the conversion of polyunsaturated compounds to the corresponding alkenes while avoiding their complete saturation and hence the formation of the corresponding alkanes.

The present invention accordingly provides a method for selective hydrogenation of a C2 steam cracking fraction comprising acetylene, said method being carried out in gaseous phase at a temperature of between 0° C. and 300° C., at a pressure of between 0.1 MPa and 6.0 MPa, at a molar ratio of hydrogen/(polyunsaturated compounds for hydrogenation) of between 0.5 and 1000, and at an hourly space velocity (HSV) of between 100 h⁻¹ and 60 000 h⁻¹, preferably between 500 h⁻¹ and 50 000 h⁻¹, in the presence of a catalyst comprising, preferably consisting of, an active phase based on at least one group VIII metal and a support provided in the form of a ceramic or metal monolith, said support comprising a number of channels per unit length (CPSI) of between 300 and 1200, and the active phase being provided in the form of a layer on the walls of said support, the thickness of said layer of active phase being between 30 μm and 150 μm.

The content of acetylene in the C2 steam cracking fraction is advantageously between 0.1% to 5% by weight with respect to the total weight of the feedstock, preferably between 0.5% and 2.5% by weight of acetylene. The C2 steam cracking fraction used for the implementation of the selective hydrogenation method according to the invention comprises, for example, the following composition: between 40% and 95% by weight of ethylene and between 0.1% to 5% by weight of acetylene, the remainder being ethane and/or methane. In some C2 steam cracking fractions, between 0.1% and 1% by weight of C3 compounds may also be present.

The selective hydrogenation method according to the invention is targeted at removing the acetylene from said feedstock for hydrogenation, without hydrogenating the monounsaturated hydrocarbons, i.e., ethylene. The technological implementation of the selective hydrogenation method is, for example, carried out by injection, in upflow or downflow mode, of the polyunsaturated hydrocarbon feedstock and of the hydrogen into at least one fixed bed reactor. Said reactor can be of isothermal type or of adiabatic type. An adiabatic reactor is preferred. The polyunsaturated hydrocarbon feedstock can advantageously be diluted by one or more reinjections of the effluent, resulting from said reactor where the selective hydrogenation reaction takes place, at one or more points of the reactor, located between the inlet and the outlet of the reactor, in order to limit the temperature gradient in the reactor. The technological implementation of the selective hydrogenation method according to the invention can also advantageously be carried out by the implantation of said catalyst in a reactive distillation column or in reactors-exchangers or in a slurry-type reactor. The stream of hydrogen can be introduced at the same time as the feedstock to be hydrogenated and/or at one or more different points of the reactor.

The selective hydrogenation of the C2 steam cracking fraction is carried out in gaseous phase. Generally speaking, the selective hydrogenation of the C2 steam cracking fraction takes place at a temperature of between 0° C. and 300° C., preferably between 15° C. and 280° C., at a pressure of between 0.1 MPa and 6.0 MPa, preferably between 0.2 MPa and 5.0 MPa, at a molar ratio of hydrogen/(polyunsaturated compounds for hydrogenation) of between 0.5 and 1000, preferably between 0.7 and 800, and at an hourly space velocity (HSV) of between 100 h⁻¹ and 60 000 h⁻¹, preferably between 500 h⁻¹ and 50 000 h⁻¹.

### Description of the Catalyst

The catalyst used within the method for selective hydrogenation comprises, and preferably consists of, an active phase based on at least one group VIII metal and a support provided in the form of a ceramic or metal monolith, characterized in that said support comprises a number of channels per unit length (CPSI) of between 300 and 1200, and in that the active phase is provided in the form of a layer on the walls of said support, the thickness of said layer of active phase being between 30 μm and 150 μm.

Preferably, the number of channels per unit length (CPSI) of said support is between 300 and 1200, preferably between 350 and 1000, more preferably between 400 and 700, and more preferably still between 450 and 750.

Preferably, the geometric surface area of said catalyst is between 1500 m²/m³ and 5000 m²/m³, preferably between 1500 m²/m³ and 4000 m²/m³, and more preferably still between 2000 m²/m³ and 4000 m²/m³.

Preferably, the thickness of the wall of the catalyst is between 0.08 mm and 0.5 mm, more preferably between 0.1 mm and 0.4 mm.

Preferably, the degree of porosity of said catalyst is between 15% and 90%, preferably between 20% and 90%, and more preferably between 20% and 70%.

Preferably, the thickness of said layer of active phase is between 60 μm and 100 μm, and more preferably still between 60 μm and 90 μm.

When the support of the catalyst is provided in the form of a metal monolith, said monolith is preferably chosen from monoliths made of steel, stainless steel (316L, 310SS), nickel, aluminum, iron, copper, nickel-chromium, nickel-chromium-aluminum, iron-chromium-aluminum, and Inconel®.

When the support of the catalyst is provided in the form of a ceramic monolith, said monolith is preferably chosen from monoliths made of alumina (Al₂O₃), silica-alumina, silicon carbide (SiC), phosphorus-alumina, magnesia (MgO), zinc oxide, zirconium oxide (ZrO₂), and cordierite (Al₃Mg₂AlSi₅O₁₈). Preferably, said ceramic monolith is made of alumina (Al₂O₃), silica-alumina, phosphorus-alumina or silicon carbide (SiC).

The group VIII metal of the active phase is preferably chosen from nickel, platinum, and palladium. Preferably, the group VIII metal is palladium.

When the group VIII metal is palladium, the palladium content is generally between 0.005% and 2% by weight of said element with respect to the total weight of the catalyst, preferably between 0.01% and 2% by weight, and more preferentially between 0.05% and 1% by weight, with respect to the total weight of the catalyst.

The catalyst can additionally comprise, as active phase, an element from Group IB, preferably chosen from silver and copper. Preferably, the element from Group IB is silver. The content of element from Group IB is preferably between 0.01% and 0.3% by weight with respect to the total weight of the catalyst, more preferably between 0.015% and 0.2% by weight.

The deposition of the active phase of the catalyst on the support provided in the form of a monolith can be carried out by conventional methods well known to the person skilled in the art, and is carried out in particular by washcoating. This impregnation technique is carried out by completely immersing the support in ceramic or metal monolith form in a solution containing the precursor salt(s) of the desired active phase(s) and then taking the said impregnated monolith out again subsequently for drying under air (preferably a stream of air). The operation can be repeated several times. The precursor of the catalyst is generally dried at a temperature of between 50° C. and 550° C., more preferably between 70° C. and 200° C. The duration of the drying is generally between 0.5 hour and 20 hours. This preparation route is carried out so as to obtain a layer of active phase on the walls of the support, the thickness of said layer being between 30 μm and 150 μm, preferentially between 60 μm and 100 μm, and more preferentially still between 60 μm and 90 μm.

### Employment of the Catalyst

In one embodiment according to the invention, the catalyst can be used in a catalytic bed in a selective hydrogenation reactor in the form of blocks of elements of cubic or parallelepipedal shape packed on top of one another. At the wall of the reactor, the monolithic support plus catalyst blocks may have a rounded shape to conform effectively to the shape of the reactor.

The selective hydrogenation reactor used in the context of the process according to the invention can be equipped with a plurality of tubes filled with the catalyst as described above. The tubes can have a circular, square or rectangular section. The wall of the tubes can be porous or nonporous. The maximum spacing between the tubes is between 0 and 100 mm, preferentially between 0 and 20 mm. According to this embodiment, the height of the reaction section can be composed of a plurality of tubes connected to one another.

The selective hydrogenation reactor used in the context of the process according to the invention can be of the reactor-exchanger type. The reactor-exchanger is equipped with a multitude of tubes filled with the catalyst as described above. The tubes can have a circular, square or rectangular section. A heat transfer fluid circulates between the tubes in order to dissipate the heat generated by the exothermic selective hydrogenation reactions. The direction of flow of the heat transfer fluid can be in the same direction as or in the opposite direction to the flow of the feedstock in the tubes. The countercurrent direction remains the preferred embodiment. The heat transfer fluid can be a liquid or a vapor which condenses.

### EXAMPLES

To illustrate some advantages of the present invention, it is proposed that the results are compared in the selective hydrogenation of acetylene using:
catalyst A (compliant): a catalyst based on palladium on an alumina support (S_{BET}=10 m²/g) which is provided in the form of beads with a diameter of 3.8 mm, the palladium content being 800 ppm by weight of Pd element with respect to the total weight of the catalyst;
catalyst B (non-compliant): a catalyst based on palladium on a support in ceramic monolith form, having geometric characteristics which are not compliant with the invention (cf. table 1 below);
catalyst C (compliant): a catalyst based on palladium on a support provided in monolith form compliant with the invention (cf. table 1 below);
catalyst D (non-compliant): a catalyst based on palladium on a support in ceramic monolith form, having geometric characteristics which are not compliant with the invention (cf. table 1 below);
catalyst E (non-compliant): a catalyst based on palladium on a support in ceramic monolith form, having geometric characteristics and the thickness of the active layer which are not compliant with the invention (cf. table 1 below).

For the catalysts B to E, the palladium active phase was deposited by the washcoating technique at a desired concentration in order to obtain, on the final catalyst, a content of palladium element of: B: 0.028% Pd, C: 0.042% Pd, D: 0.054% Pd, and E: 0.015% Pd by weight with respect to the total weight of the catalyst.

TABLE 1

| Catalyst | A | B | C | D | E |
|---|---|---|---|---|---|
| CPSI of the monolith | — | 200 | 600 | 2500 | 2500 |
| Window width (mm) | — | 1.32 | 0.7 | 0.22 | 0.22 |
| Thickness of the | — | 90 | 90 | 90 | 20 |
| active phase layer | | | | | |
| (μm) | | | | | |
| Geometric surface | 780 | 1600 | 2600 | 3500 | 5700 |
| area of the catalyst | | | | | |
| (m²/m³) | | | | | |
| Degree of porosity | 42 | 53 | 49 | 19 | 31 |
| (%) | | | | | |

The operating conditions considered are given in table 2. They are identical for the five cases studied.

TABLE 2

| | |
|---|---|
| Total throughput by mass (tonnes/hour) | 39.6 |
| T (° C.) | 65 |
| P (MPa) | 2 |
| Acetylene content in the feedstock (ppm) | 2500 |
| Ethane content in the feedstock (ppm) | 200 |
| Ethylene content in the feedstock (mol/mol) | 0.5 |
| Hydrogen content in the feedstock (mol/mol) | 0.16 |
| CO content in the feedstock (ppm) | 200 |
| Propane content in the feedstock (mol/mol) | 0.3371 |
| H₂/C₂H₂ (mol/mol) | 64 |

The results are given in table 3 below. For all of these results, a reactor 1 m in diameter is used.

TABLE 3

| | | Content of | | | | | | |
|---|---|---|---|---|---|---|---|---|
| | | acetylene | Selectivity | | | | | |
| | Conversion | at the | for | Total | Total | Reactor | Volume of | |
| | Acetylene | outlet | ethylene | ΔT | ΔP | length | reactor | HSV |
| Catalyst | (%) | (ppm) | (%) | (° C.) | (MPa) | (m) | (m³) | (h⁻¹) |
| A | 99.95 | 1 | 59.8 | 9.8 | 0.27 | 1.91 | 1.5 | 30000 |
| B | 99.95 | 1.3 | 64.5 | 9.3 | 0.004 | 1.91 | 1.5 | 30000 |
| C | 99.96 | 1 | 63.4 | 9.4 | 0.009 | 1.32 | 1.03 | 43000 |
| D | 99.96 | 1 | 61.9 | 9.7 | 0.197 | 1.0 | 0.78 | 57000 |
| E | 99.43 | 14 | 79.8 | 8.5 | 0.23 | 1.91 | 1.5 | 30000 |

It is seen that all of the catalysts have a conversion of more than 99% of the acetylene, with an acetylene content at the reactor outlet of 1 ppm, or even of more than 1 ppm for catalyst B not compliant with the invention (1.3 ppm) and for catalyst E not compliant with the invention (14 ppm). This conversion is achieved with a reduction in reactor volume when the method is carried out in the presence of catalysts C (compliant) and D (non-compliant) in relation to a method in the presence of a catalyst A (non-compliant) having a support provided in the form of beads. The use of catalyst C according to the invention also enables a reduction in reactor volume of 30% by volume equally in relation to the non-compliant catalysts A and B, and a gain in terms of pressure drop. At the same conversion, then, the total volume of the reactor can be reduced. Also noted is the impact of the selection of channel density (CPSI) of the support on the method according to the invention. The reason is that catalysts B and D, not compliant with the invention, although provided in the monolith form, have mediocre results in terms both of acetylene content at the reactor outlet (whereas the reactor has a higher catalyst volume) for the catalyst B and of pressure drop for the catalyst D (and a little in terms of selectivity). Lastly, the non-compliant catalyst E, though exhibiting a higher selectivity than catalyst C according to the invention, has a poorer conversion (and hence too high an acetylene content at the reactor outlet) with an increased pressure drop, owing respectively to an excessive channel density and to a low active phase layer thickness. It is therefore only catalyst C according to the invention that enables a trade-off between acetylene selectivity, pressure drop, and catalytic reaction volume.

1. A method for selective hydrogenation of a C2 steam cracking fraction comprising acetylene, said method comprising:
performing selective hydrogenation of said C2 steam cracking fraction in gaseous phase at a temperature of between 0° C. and 300° C., at a pressure of between 0.1 MPa and 6.0 MPa, at a molar ratio of hydrogen/(polyunsaturated compounds for hydrogenation) of between 0.5 and 1000, and at an hourly space velocity of between 100 h⁻¹ and 60 000 h⁻¹, in the presence of a catalyst comprising an active phase based on at least one group VIII metal and a support provided in the form of a ceramic or metal monolith,
wherein said support comprises a number of channels per unit length, CPSI, of between 300 and 1200, and in that the active phase is provided in the form of a layer on walls of said support, the thickness of said layer of active phase being between 30 μm and 150 μm.

2. The method as claimed in claim 1, in which said catalyst comprises a geometric surface area of between 1500 m²/m³ and 5000 m²/m³.

3. The method as claimed claim 1, in which the wall thickness of the catalyst is between 0.08 mm and 0.5 mm.

4. The method as claimed in claim 1, in which said catalyst has a degree of porosity of between 20% and 90%.

5. The method as claimed in claim 1, in which the thickness of said layer of active phase is between 60 μm and 100 μm.

6. The method as claimed in claim 1, in which the support is a ceramic monolith chosen from monoliths made of alumina (Al₂O₃), silica-alumina, silicon carbide (SiC), phosphorus-alumina, magnesia (MgO), zinc oxide, zirconium oxide (ZrO₂) or cordierite (Al₃Mg₂AlSi₅O₁₈).

7. The method as claimed in claim 1, in which said group VIII metal is chosen from nickel, platinum, and palladium.

8. The method as claimed in claim 7, in which said group VIII metal is palladium.

9. The process as claimed in claim 8, in which the palladium content is between 0.005% and 2% by weight with respect to the total weight of the catalyst.

10. The method as claimed in claim 1, in which the number of channels per unit length of said support is between 400 and 700.
